(19) United States
(12) Patent Application Publication
(10) Pub. No.: US 2017/0298128 A1
(43) Pub. Date: Oct. 19, 2017

(54) METHODS AND COMPOSITIONS FOR TREATING AGE-RELATED DISORDERS

(71) Applicants: President and Fellows of Harvard College, Cambridge, MA (US); The Brigham and Women's Hospital, Inc., Boston, MA (US)

(72) Inventors: Thomas M. Barnes, Brookline, MA (US); Amy J. Wagers, Cambridge, MA (US); Richard T. Lee, Weston, MA (US)

(21) Appl. No.: 15/513,979

(22) PCT Filed: Nov. 23, 2015
(86) PCT No.: PCT/US15/62226

§ 371 Date: Mar. 23, 2017

Related U.S. Application Data
(60) Provisional application No. 62/053,956, filed on Sep. 23, 2014.

(51) Int. Cl. C07K 16/28; C07K 16/22

(52) U.S. Cl. CPC C07K 16/28; C07K 16/22; C07K 2317/21; C07K 2317/76; C07K 2317/24

(57) ABSTRACT

The present invention relates to methods and compositions containing an antagonist of a negative regulator of GDF-11 (e.g., an antibody) for use in treating an age-related condition. In particular the methods and compositions can be used to treat an age related cardiovascular condition such as diastolic heart failure.

### RELATED APPLICATION

This application claims the benefit of U.S. Provisional Application Ser. No. 62/053,956, filed Sep. 23, 2014, the entire teachings of which are incorporated herein by reference.

### BACKGROUND

In complex organisms, aging consists of global and organ-specific changes at the molecular, tissue and macroscopic levels. Not only are there the well-known manifestations in hair color and skin elasticity, but there are many others, such as the regenerative capacity of muscle, an increase in circulating cholesterol, the activity of the neural stem cell niche and changes in endocrine function. For therapeutic purposes, there is an important distinction between age-dependent processes that lead to a decline in organ or organismal function, and diseases whose incidence is age-dependent. A common presumption is that the molecular processes that underlie age-dependent decline in function also underlie the pathophysiology of age-dependent diseases. For some diseases, such as cardiac dysfunction, this is reasonable since the initial reaction of cardiomyocytes to stress or damage is adaptive, and is a normal physiological response. Only with a prolonged adaptive response does dysfunction arise, and this dysfunction is similar in young or old mammals under chronic cardiac stress. In other diseases, the loss or lack of regenerative processes sets up irreversible decline over time. In these cases, what is required are therapies that can re-animate regenerative processes, such as those that control the behavior of organ-specific stem cells. Agents that regulate the proliferative capacity of stem cells could thus be useful to treat diseases in non-regenerative tissues, such as the CNS and muscle.

As mentioned above, aging in multicellular organisms can lead to the loss of normal cardiac function, ultimately resulting in heart failure. Heart failure affects approximately 1% of individuals over 50 but over 5% of individuals over 75. With the ongoing steep rise in the proportion of elderly individuals within our population, age-related heart failure is certain to become an increasingly prevalent health condition. Most age-related heart failure is in the setting of normal systolic function, and this is a condition often associated with cardiac hypertrophy (i.e., enlargement of heart tissue) and called “diastolic heart failure” (Aurigemma (2006) N. ENGL. J. MED. 355:308). Diastolic heart failure accounts for 40-60% of heart failure cases (Aurigemma, supra; Hunt et al. (2009) CIRCULATION 119:e391; Kitzman et al. (2007) CLIN. GERIATR. MED. 23:83-106). The prognosis of diastolic heart failure may be as poor as systolic heart failure (Aurigemma, supra), with a 5-year risk of death after an initial heart failure hospitalization approaching that of common malignancies (Wright et al. (2001) SCIENCE 294:1933-1936). Although much progress has been made in the treatment of systolic heart failure, with substantial improvements in outcome over the past two decades, progress in treatment of diastolic heart failure has been much more elusive (Hunt et al. (2009) CIRCULATION 119:e391-479). Indeed, one can argue that there are no specific therapies for patients who experience the ventricular “stiffening” associated with the diastolic dysfunction that accompanies aging (Kitzman et al., supra). This may explain the observation that mortality is declining for systolic heart failure but not diastolic heart failure and underscores the enormous clinical demand for new therapeutic strategies targeting diastolic failure.

Previous work has shown that the amount of growth differentiation factor 11 (GDF-11) is decreased in older mice, the lower amount of GDF-11 is associated with cardiac hypertrophy, and that the administration of GDF-11 can reverse age-related cardiac hypertrophy (Loffredo et al. (2013) CELL 153:828-839 and PCT Publication No. WO 2013/142114), which can be beneficial in treatment of diastolic heart failure. Nevertheless, there is still a need for alternative approaches for treating age-related conditions and disorders.

### SUMMARY

The present invention is based, in part, upon the understanding that a supra-physiological dose of GDF-11, via down-stream signaling pathways, can impart an undesirable effect in a variety of different tissues, such as atrophy in skeletal muscle. Furthermore, GDF-11, as a small soluble cytokine, likely has a short half-life of typically less than 1 hour. As a result, the systemic administration of GDF-11 may need to occur within a tight dosing range or concentration and/or dosing regimen, potentially limiting its therapeutic utility. As a result, tan approach has been developed for increasing GDF-11 activity with an agent that has a prolonged half-life and in a manner that avoids supra-physiological GDF-11 in a subject, making administration safer and more convenient.

The approach is based upon antagonism of the effect of an endogenous negative regulator (for example, inhibitor) of GDF-11 activity. As a result, GDF-11 activity can be increased in a desired tissue where the negative regulator is present, or in circulation in general, but without the problems associated with systemic administration of GDF-11. This approach results in a safe and convenient enhancement of GDF-11 activity that can be useful in treating subjects suffering from an age-related condition, particularly diastolic heart failure.

Accordingly, in a first aspect, the invention features a method of treating an age-related condition in a subject in need thereof. The method includes administering to the subject an effective amount of an antagonist of an endogenous negative regulator of GDF-11 activity, thereby ameliorating at least one symptom of the age-related condition (e.g., a cardiovascular disorder, a cognitive disorder, a neurodegenerative disorder, a metabolic disorder, or a muscular disorder).

In another aspect, the invention features a method of ameliorating at least one symptom of diastolic heart failure in a subject in need thereof. The method includes administering to the subject an effective amount of an antagonist of an endogenous negative regulator of GDF-11 activity, thereby ameliorating at least one symptom of diastolic heart failure in the subject. In certain embodiments, the subject has preserved ejection fraction but elevated left ventricular diastolic pressure (LVDP), as compared to subjects without diastolic heart failure. In certain embodiments, the subject has preserved ejection fraction but elevated myocardial fibrosis, as compared to subjects without diastolic heart failure. Administration of the antagonist can reduce left ventricle wall thickness, myocardial fibrosis, or both left ventricle wall thickness and myocardial fibrosis in the subject. The myocardial fibrosis may be caused by an accumulation of extracellular matrix fibrillar collagen, and the administration of the antagonist reduces the amount of extracellular matrix fibrillar collagen in the subject relative to before initial administration of the antagonist.

In another aspect, the invention features a method of treating diastolic heart failure in a subject in need thereof. The method includes administering an effective amount of an antagonist of an endogenous negative regulator of GDF-11 activity to the subject, thereby treating diastolic heart failure in the subject. The antagonist reduces left ventricle wall thickness in the subject relative to left ventricle wall thickness prior to initial administration of the antagonist. In certain embodiments, the administration of the antagonist reduces myocardial fibrosis in the subject relative to myocardial fibrosis prior to initial administration of the antagonist. The myocardial fibrosis may be caused by an accumulation of extracellular matrix fibrillar collagen, and the administration of the antagonist may reduce the amount of extracellular matrix fibrillar collagen in the subject relative to before initial administration of the antagonist.

In any of the above aspects, the endogenous negative regulator of GDF-11 activity can be follistatin (FS; see, e.g., NCBI Reference Sequences: NP_037541.1, NP_006341.1, XP_005248460.1, XP_005248459.1, XP_005248458.1, and XP_005248457.1), follistatin-like 3 (FSTL3, also known as follistatin related gene, or FLRG; see, e.g., NCBI Reference Sequence: NP_005851.1), GDF-associated serum protein-1 (GASP1, also known as WFIKKN2 or WFIKKN-related protein; see, e.g., NCBI Reference Sequence: NP_783165.1), GASP2 (also known as WFIKKN1, WFIKKN, or C16orf12; see, e.g., NCBI Reference Sequence: NP_444514.1), the GDF-11 propeptide, and the myostatin propeptide. In any of the above aspects, the antagonist can be a protein, for example, an antibody such as an anti-FS antibody, an anti-FSTL3 antibody, an anti-GASP1 antibody, an anti-GASP2 antibody, an anti-GDF-11 propeptide antibody, or an anti-myostatin propeptide antibody. In any of the above aspects, the activity of the endogenous regulator that is negatively affected by the antagonists is the regulator's ability to bind mature or latent GDF-11 proteins. In specific embodiments, the antagonist is an anti-GASP1 antibody. In specific embodiments, the antagonist is an anti-GASP2 antibody. In specific embodiments, the antagonist is an anti-FS antibody.

In any of the above aspects, the antagonist, in certain embodiments, does not cause a substantial (i) reduction of skeletal muscle mass of the subject, (ii) reduction of erythropoiesis in the subject, (iii) increase in follicle-stimulating hormone (FSH) activity in the subject, or (iv) a combination thereof. In particular embodiments, the administration of the antagonist does not cause anosmia in the subject.

In another aspect, the invention features a method of reducing cardiomyocyte cell size. The method includes exposing a viable cardiomyocyte to an antagonist selected from the group consisting of a GASP1 antagonist, a GASP2 antagonist, and a FS antagonist, in an amount sufficient to cause a reduction in cardiomyocyte cell size relative to cardiomyocyte cell size prior to exposure to the antagonist. In one embodiment, the antagonist is an anti-GASP1 antibody. In another embodiment, the antagonist is an anti-GASP2 antibody. In another embodiment, the antagonist is an anti-FS antibody.

These and other aspects and advantages of the invention will become apparent upon consideration of the following figures, detailed description, and claims. A s used herein, “including” means without limitation, and examples cited are non-limiting.

### DESCRIPTION OF THE DRAWINGS

The invention can be more completely understood with reference to the following drawings.

FIG. 1 is a schematic diagram showing the physiological regulation of GDF-11 activity, and the proposed points of inhibitor intervention.

### DETAILED DESCRIPTION

The present invention is based on the development of new approaches for the treatment of age-related disorders, particularly diastolic heart failure, by inhibiting endogenous negative regulators of GDF-11, including follistatin (FS), follistatin-like 3 (FSTL3), GDF-associated serum protein-1 (GASP1), GASP2, the GDF-11 propeptide, and the myostatin propeptide.

Briefly, the invention is based on the identification of a strategy for increasing GDF-11 activity in cells, tissues, organs, or body fluids by inhibiting endogenous negative regulators of GDF-11 that are naturally present in cells, tissues, or fluids (e.g., blood). In contrast to simply administering GDF-11 to a subject or globally increasing GDF-11 expression, selective inhibition of a particular GDF-11 negative regulator can prevent supra-physiological levels of GDF-11 entering circulation. This can be advantageous for decreasing side effects and can allow for higher doses of the antagonist or increased localized concentrations of active GDF-11 in the desired tissues with smaller or no increases activity elsewhere, depending on the site of action of the endogenous regulator.

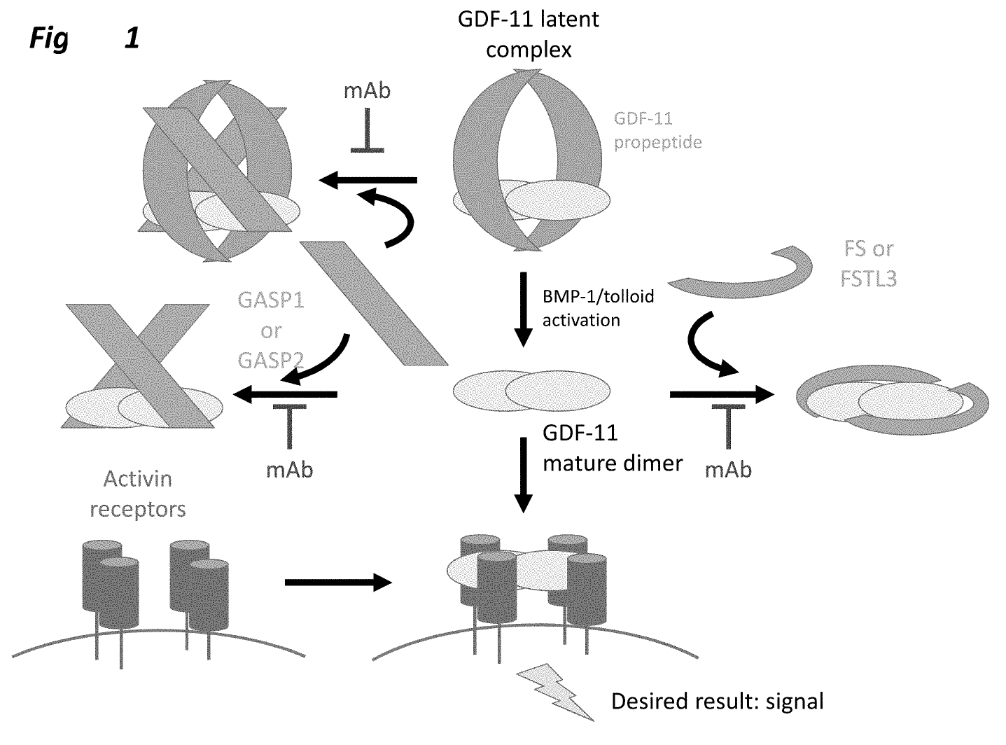

Possible sites for therapeutic intervention are shown schematically in FIG. 1. GDF-11 is secreted as a latent complex (see top of FIG. 1), which includes a GDF-11 dimer bound to the GDF-11 propeptide. To activate GDF-11, the propeptide is enzymatically cleaved to free the mature dimer (see center of FIG. 1), which can then bind its cognate receptor on a target cell. Both mature and latent GDF-11 can be neutralized by soluble secreted regulators. For example FS and FSTL3 only binds mature GDF-11, while GASP1 and GASP2 can bind both mature and latent GDF-11. Inhibitor activity can be blocked at the points indicated by “mAb,” although it is apparent that a variety of inhibitors, for example, antibodies, for example, monoclonal antibodies (mAb), small molecules, aptamers, can be used in the practice of the invention.

As used herein, the term an “age-related condition” refers to any disease, disorder, or undesirable state whose incidence in a population or severity in an individual correlates with the progression of age. In some embodiments, the age-related condition is a cardiovascular condition, aging of the heart, aging of skeletal muscle, or aging of the brain. Aging of any given organ can include, but is not limited to, reduced cellularity, reduced stem cell genomic integrity, reduced cellular function (e.g., reduced muscle contraction in muscle tissue), reduced regenerative capacity, atrophy (e.g., aging of the skin can include atrophy of the epidermis and/or sebaceous follicles). An age-related condition can be one that reduces the function of a given organ or one that is aesthetically undesirable (e.g., aging of the skin or muscle can be aesthetically undesirable). Additional age-related conditions can include, but are not limited to, sarcopenia, skin atrophy, muscle wasting, brain atrophy, atherosclerosis, arteriosclerosis, pulmonary emphysema, osteoporosis, osteoarthritis, immunologic incompetence, high blood pressure, dementia, Huntington's disease, Alzheimer's disease, cataracts, age-related macular degeneration, prostate cancer, stroke, diminished life expectancy, memory loss, wrinkles, impaired kidney function, and age-related hearing loss.

As used herein, the term “cardiovascular condition” or “cardiovascular disorder” refers to a heart or circulatory system condition or disorder mediated or characterized by a reduction in circulating GDF-11 polypeptide or GDF-11 activity. Non-limiting examples of cardiovascular conditions include diastolic heart failure, cardiac hypertrophy, hypertension, valvular disease, aortic stenosis, genetic hypertrophic cardiomyopathy, or stiffness of the heart due to aging.

“Metabolic disorder,” as used herein, shall mean any disease or disorder that damages or interferes with normal function in a cell, tissue, or organ by affecting the production of energy in cells or the accumulation of toxins in a cell, tissue, organ, or individual. Metabolic disorders include, but are not limited to, type II diabetes, metabolic syndrome, hyperglycemia, and obesity.

As used herein, an “endogenous negative regulator of GDF-11” is understood to mean any molecule that occurs naturally in an organism and is capable of inhibiting GDF-11 signaling activity. This is typically accomplished by binding and/or sequestering GDF-11. Exemplary endogenous negative regulators of GDF-11 include FS, FSTL3, GASP1, GASP2, GDF-11 propeptide, and myostatin propeptide.

As used herein, the term “GDF-11” is meant the mature, physiological active form of GDF-11, which in humans is typically a 109 amino acid protein that is produced by cleavage of the precursor protein. The “GDF-11 propeptide” refers to the N-terminal portion of the precursor protein that is produced by cleavage of the precursor. The GDF-11 propeptide is capable of binding and inhibiting GDF-11 activity. Upon synthesis and release from the cell, the GDF-11 propeptide remains stably associated with GDF-11, to form the “GDF-11 latent complex”. The GDF-11 propeptide must be cleaved by the metalloproteases of the BMP-1/tolloid family to release the signaling-competent form of GDF-11. The precursor protein sequence of GDF-11 sequence is found, for example, in NCBI Reference Sequence NP_005802.1.

In one aspect, the invention provides a method of treating an age-related condition in a subject in need thereof. The method includes administering to the subject an effective amount of an antagonist of an endogenous negative regulator of GDF-11 activity, thereby ameliorating at least one symptom of the age-related condition (e.g., a cardiovascular disorder, a cognitive disorder, a neurodegenerative disorder, a metabolic disorder, or a muscular disorder).

As used herein, an “antagonist of an endogenous negative regulator of GDF-11 activity” is understood to mean an agent that acts by reducing the expression of the endogenous negative regulator, or by antagonizing the ability of the endogenous negative regulator to bind to either GDF-11 or the GDF-11 latent complex.

As used herein, the term “treat,” “treating,” and “treatment” is understood to mean the treatment of a disease in a mammal, e.g., in a human. This includes (a) reducing at least one symptom associated with the disease, (b) inhibiting the disease, i.e., arresting its development, and (c) relieving the disease, i.e., causing regression of the disease state.

In another aspect, the invention provides a method of ameliorating at least one symptom of diastolic heart failure in a subject in need thereof. The method includes administering to the subject an effective amount of an antagonist of an endogenous negative regulator of GDF-11 activity, thereby ameliorating at least one symptom of diastolic heart failure in the subject. In certain embodiments, the subject has preserved ejection fraction but elevated left ventricular diastolic pressure (LVDP), as compared to subjects without diastolic heart failure. In certain embodiments, the subject has preserved ejection fraction but elevated myocardial fibrosis, as compared to subjects without diastolic heart failure. Administration of the antagonist can reduce left ventricle wall thickness, myocardial fibrosis, or both left ventricle wall thickness and myocardial fibrosis in the subject. The myocardial fibrosis may be caused by an accumulation of extracellular matrix fibrillar collagen, and the administration of the antagonist reduces the amount of extracellular matrix fibrillar collagen in the subject relative to before initial administration of the antagonist.

In another aspect, the invention provides a method of treating diastolic heart failure in a subject in need thereof. The method includes administering an effective amount of an antagonist of an endogenous negative regulator of GDF-11 activity to the subject, thereby treating diastolic heart failure in the subject. The antagonist reduces left ventricle wall thickness in the subject relative to left ventricle wall thickness prior to initial administration of the antagonist. In certain embodiments, the administration of the antagonist reduces myocardial fibrosis in the subject relative to myocardial fibrosis prior to initial administration of the antagonist. The myocardial fibrosis may be caused by an accumulation of extracellular matrix fibrillar collagen, and the administration of the antagonist may reduce the amount of extracellular matrix fibrillar collagen in the subject relative to before initial administration of the antagonist.

In any of the above aspects, the endogenous negative regulator of GDF-11 activity can be follistatin (FS), follistatin-like 3 (FSTL3), GDF-associated serum protein-1 (GASP1), GASP2, the GDF-11 propeptide, and the myostatin propeptide. In any of the above aspects, the antagonist can be a protein, for example, an antibody such as an anti-FS antibody, an anti-FSTL3 antibody, an anti-GASP1 antibody, an anti-GASP2 antibody, an anti-GDF-11 propeptide antibody, or an anti-myostatin propeptide antibody. In specific embodiments, the antagonist is an anti-GASP1 antibody. In specific embodiments, the antagonist is an anti-GASP2 antibody. In specific embodiments, the antagonist is an anti-FS antibody.

As used herein, unless otherwise indicated, “antibody” means an intact antibody (e.g., an intact monoclonal antibody) or antigen-binding fragment of an antibody, including an intact antibody or antigen-binding fragment that has been modified or engineered, or that is a human antibody. Examples of antibodies that have been modified or engineered are chimeric antibodies, humanized antibodies, and multispecific antibodies (e.g., bispecific antibodies). Examples of antigen-binding fragments include Fab, Fab′, F(ab′)₂, Fv, single chain antibodies (e.g., scFv), minibodies, and diabodies.

In any of the above aspects, the antagonist, in certain embodiments, does not cause a substantial (i) reduction of skeletal muscle mass of the subject, (ii) reduction of erythropoiesis in the subject, (iii) increase in follicle-stimulating hormone (FSH) activity in the subject, or (iv) a combination thereof. In particular embodiments, the administration of the antagonist does not cause anosmia in the subject.

In another aspect, the invention provides a method of reducing cardiomyocyte cell size. The method includes exposing a viable cardiomyocyte to an antagonist selected from the group consisting of a GASP1 antagonist, a GASP2 antagonist, and a FS antagonist, in an amount sufficient to cause a reduction in cardiomyocyte cell size relative to cardiomyocyte cell size prior to exposure to the antagonist. In one embodiment, the antagonist is an anti-GASP1 antibody. In another embodiment, the antagonist is an anti-GASP2 antibody. In another embodiment, the antagonist is an anti-FS antibody.

### GDF-11 and GDF-11 Signaling

Human GDF-11, also known as bone morphogenetic protein 11 (BMP-11), is translated as a 407 amino acid polypeptide. A 24 amino acid amino-terminal signal sequence is cleaved from this polypeptide, thereby forming a 383 amino acid precursor polypeptide. The precursor polypeptide is cleaved to form a carboxy-terminal 109 amino acid active polypeptide and an amino-terminal 274 amino acid polypeptide termed the “propeptide,” which as described below, inhibits GDF-11 activity. Upon synthesis, the propeptide remains stably associated with the active GDF-11, to form the “GDF-11 latent complex”. The latent complex comprises a dimer of active GDF-11 covalently joined via a disulphide bond, each monomer of which is bound non-covalently to one propeptide. The GDF-11 propeptide must be cleaved by the metalloproteases of the BMP-1/tolloid family to release the signaling-competent form of GDF-11. The active carboxy-terminal polypeptide forms a homodimer through disulfide bonds (see, e.g., McPherron (2010) IMMUNOL. ENDOCR. METAB. AGENTS MED. CHEM. 10:217-231).

GDF-11 is closely related to myostatin (also known as GDF-8). The mature form of GDF-11 shares 89% sequence identity with the mature form of myostatin. The activities of the two proteins are indistinguishable in vitro, but different expression patterns in vivo appear to affect their respective biological functions (Lee et al. (2013) PROC. NATL. ACAD. SCI. USA 110:E3713-3722).

The active form of GDF-11 is an agonist of activin receptors, including ActRIIA and ActRIIB as the type II receptors, and ALK4, ALK5 and ALK7 as the type I receptors (Oh et al. (2002) GENES DEV. 16:2749-2754). Based on ActRII knockout phenotypes, ActRIIB is the main type II receptor for GDF-11 in mice. Activation of this receptor activates Smad 2/3 by phosphorylation, thereby blocking cell cycle progress and altering cell fate. While GDF-11 plays important roles during embryonic development, it also has been shown to play a role in age-related disorders, including cardiac hypertrophy. See, for example, PCT Publication No. WO 2013/142114, which describes increasing GDF-11 levels to treat diastolic heart failure. The present invention, by contrast, relies instead on increasing GDF-11 activity, not by administering GDF-11, but by increasing the activity of endogenous GDF-11, as described below.

### Endogenous Negative Regulators of GDF-11

In addition to being produced in a latent complex, GDF-11 activity is controlled by a number of endogenous negative regulators. These negative regulators include FS, FSTL3, GASP1, GASP2, the GDF-11 propeptide, and the myostatin propeptide.

Follistatin and follistatin-like 3 (also called follistatin-like gene protein) have been identified as negative regulators of GDF-11 in several contexts (Tsuchida et al. (2009) CELL COMMUN. SIGNAL. 7:15). Follistatin, in particular has been identified as regulating GDF-11 in olfactory epithelium, and muscle cells (Wu et al. (2003) NEURON 37:192-208).

GASP1 and GASP2 have also been identified as negatively regulating GDF-11. These proteins both can act by inhibiting binding of GDF-11 or the latent GDF-11 complex to its high-affinity receptor, the activin type IIB receptor (Lee et al., supra).

The GDF-11 propeptide discussed above (i.e., the 274 amino acid amino-terminal fragment of the GDF-11 precursor) has been shown to bind and inhibit the active GDF-11 dimer in vitro. Overexpression of the propeptide in transgenic mice has shown similar but less dramatic phenotype as compared to GDF-11 null mice, thus confirming that that the propeptide has inhibitory activity in vivo (Li et al. (2010) MOL. REPROD. DEV. 77:990-997).

### Antagonistsof EndogenousNegativeRegulatorsof GDF-11

Any appropriate antagonist of an endogenous negative regulator of GDF-11 (e.g., those described herein) can be used in the context of the present invention. The antagonist can, in certain embodiments, be an antibody, a small molecule antagonist, or an aptamer. An antagonist of an endogenous negative regulator can also be a selective gene expression inhibitor, such as an antisense oligonucleotide, an RNAi molecule, or a CRISPR/Cas9 complex.

### Antibody-Based Antagonists

The methods of the invention can employ antibodies that inhibit endogenous negative regulators of GDF-11. Antibodies can be directed against FS, FSTL3, GASP1, GASP2, the GDF-11 propeptide, or the myostatin propeptide.

In some embodiments, the antibody binds the endogenous negative regulator of GDF-11 with a K_{D} of about 300 pM, 250 pM, 200 pM, 190 pM, 180 pM, 170 pM, 160 pM, 150 pM, 140 pM, 130 pM, 120 pM, 110 pM, 100 pM, 90 pM, 80 pM, 70 pM, 60 pM, 50 pM, 40 pM, 30 pM, 20 pM, or 10 pM, or lower. Unless otherwise specified, K_{D} values are determined by surface plasmon resonance methods or biolayer interferometry.

Particular examples of such antibodies include the anti-myostatin propeptide monoclonal antibodies described in Abcam catalog #ab37254 and ab37257; the anti-FS monoclonal antibodies described in Abcam catalog #ab89515, Ansh Labs catalog #Ab-307, R&D Systems catalog #MAB669, AbD Serotec catalog #MCA4736GA, Proteintech catalog #60060-1-Ig, Santa Cruz Biotechnology catalog #sc-271502 and sc-365003; the anti-FSTL3 monoclonal antibodies described in Abcam catalog #ab86055 and ab65202, Creative Biomart catalog #CABT-28407RM, Abnova catalog #MAB9381; the anti-GASP1 monoclonal antibodies described in R&D Systems catalog #MAB2070, Novus Biologicals catalog #NBP2-21976, Abbexa catalog #abx10858; and the anti-GASP2 monoclonal antibodies described in R&D Systems catalog #MAB2136, LifeSpanBiosciences catalog #LS-C36420 and LS-C36419, Creative Biomart catalog #CABT-37648MH, Abcam catalog #ab89562.

### Antibody Development

Additional antibodies (e.g., monoclonal, polyclonal, poly-specific, or mono-specific antibodies) against an endogenous negative regulator of GDF-11 can be made, e.g., using any of the numerous methods for making antibodies known in the art. In one example, a coding sequence for the negative regulator is expressed as a carboxy-terminal fusion with glutathione S-transferase (GST) (Smith et al. (1998) GENE 67:31-40). The fusion protein is purified on glutathione-Sepharose beads, eluted with glutathione, cleaved with thrombin (at an engineered cleavage site), and purified for immunization of rabbits. Primary immunizations are carried out with Freund's complete adjuvant and subsequent immunizations with Freund's incomplete adjuvant. Antibody titers are monitored by Western blot and immunoprecipitation analyses using the thrombin-cleaved protein fragment of the GST fusion protein. Immune sera are affinity purified using CNBr-Sepharose-coupled protein. Antiserum specificity can be determined using a panel of unrelated GST proteins.

As an alternative or adjunct immunogen to GST fusion proteins, peptides corresponding to relatively unique immunogenic regions of a polypeptide of the invention can be generated and coupled to keyhole limpet hemocyanin (KLH) through an introduced carboxy-terminal lysine. Antiserum to each of these peptides is similarly affinity purified on peptides conjugated to BSA, and specificity is tested by ELISA or Western blot analysis using peptide conjugates, or by Western blot or immunoprecipitation using the polypeptide expressed as a GST fusion protein.

Alternatively, monoclonal antibodies that specifically bind the endogenous negative regulator can be prepared using standard hybridoma technology (see, e.g., Kohler et al. (1975) NATURE 256:495-497; Kohler et al. (1976) EUR. J. IMMUNOL. 6:511-519; Kohler et al. (1976) EUR. J. IMMUNOL. 6:292-295; Hammerling et al., Monoclonal Antibodies and T Cell Hybridomas, Elsevier, N.Y., 1981). Once produced, monoclonal antibodies can also be screened for specific recognition by Western blot or immunoprecipitation analysis. Alternatively, monoclonal antibodies can be prepared using the polypeptide of the invention described above and a phage display library (Vaughan et al. (1996) NAT. BIOTECHNOL. 14:309-314).

Epitopic fragments can be generated by standard techniques, e.g., using PCR and cloning the fragment into a pGEX expression vector. Fusion proteins can be expressed in E. coli and purified using a glutathione agarose affinity matrix. To minimize potential problems of low affinity or specificity of antisera, two or three such fusions can be are generated for each protein, and each fusion is injected into at least two mice. Antisera are raised by injections in a series, and can include, for example, at least three booster injections.

In order to generate polyclonal antibodies on a large scale and at a low cost an appropriate animal species can be chosen. Polyclonal antibodies can be isolated from the milk or colostrum of, e.g., immunized cows. Bovine colostrum contains 28 g of IgG per liter, while bovine milk contains 1.5 g of IgG per liter (Ontsouka et al. (2003) J. DAIRY Sci 86:2005-2011). Polyclonal antibodies can also be isolated from the yolk of eggs from immunized chickens (Sarker et al. (2001) J. PEDIATR. GASTROENTEROL. NUTR. 32:19-25).

Multiple adjuvants are approved for use in dairy cows. Adjuvants useful in this invention include, but are not limited to, Emulsigen®, an oil-in-water emulsified adjuvant, Emulsigen®-D, an oil-in-water emulsified adjuvant with DDA immunostimulant, Emulsigen®-P, an oil-in-water emulsified adjuvant with co-polymer immunostimulant, Emulsigen®-BCL, an oil-in-water emulsified adjuvant with block co-polymer immunostimulant, Carbigen™, a carbomer base, and Polygen™, a co-polymer base. All of the listed adjuvants are commercially available from MVP Laboratories in Omaha, Neb.

Useful antibodies can be identified in several different screening assays. First, antibodies are assayed by ELISA to determine whether they are specific for the immunizing antigen (i.e., an endogenous negative regulator of GDF-11 described herein). Using standard techniques, ELISA plates are coated with immunogen, the antibody is added to the plate, washed, and the presence of bound antibody detected by using a second antibody specific for the Ig of the species in which the antibody was generated.

A functional in vitro assay can be used to screen antibodies e.g., an neutralizing assay based on monocyte-derived dendritic cells.

### Antibody Production

Antibodies can be produced using any methods known in the art. For example, DNA molecules encoding light chain variable regions and/or heavy chain variable regions can be chemically synthesized using the sequence information provided herein. Synthetic DNA molecules can be ligated to other appropriate nucleotide sequences, including, e.g., constant region coding sequences, and expression control sequences, to produce conventional gene expression constructs encoding the desired antibodies. Production of defined gene constructs is within routine skill in the art. Alternatively, the sequences provided herein can be cloned out of hybridomas by conventional hybridization techniques or polymerase chain reaction (PCR) techniques, using synthetic nucleic acid probes whose sequences are based on sequence information provided herein, or prior art sequence information regarding genes encoding the heavy and light chains of murine antibodies in hybridoma cells.

Nucleic acids encoding desired antibodies can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed secreted protein accumulates in refractile or inclusion bodies, and can be harvested after disruption of the cells by French press or sonication. The refractile bodies then are solubilized, and the proteins refolded and cleaved by methods known in the art.

If the engineered gene is to be expressed in eukayotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. This expression vector optionally contains sequences encoding all or part of a constant region, enabling an entire, or a part of, a heavy or light chain to be expressed. The gene construct can be introduced into eukaryotic host cells using conventional techniques. The host cells express V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments, a host cell is transfected with a single vector expressing a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain. In some embodiments, a host cell is co-transfected with more than one expression vector (e.g., one expression vector expressing a polypeptide comprising an entire, or part of, a heavy chain or heavy chain variable region, and another expression vector expressing a polypeptide comprising an entire, or part of, a light chain or light chain variable region).

A polypeptide comprising an immunoglobulin heavy chain variable region or light chain variable region can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

A monoclonal antibody that binds an endogenous negative regulator of GDF-11, or an antigen-binding fragment of the antibody, can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. The intact antibody (or antigen-binding fragment) can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

### Antibody Modifications

Methods for reducing or eliminating the antigenicity of antibodies and antibody fragments are known in the art. When the antibodies are to be administered to a human, the antibodies preferably are modified to reduce or eliminate antigenicity in humans. For example, the antibodies can be humanized antibodies or fully human antibodies. Preferably, each antibody that has been modified to reduce immunogenicity has the same or substantially the same affinity for the antigen as a non-humanized mouse antibody from which it was derived.

In one humanization approach, chimeric proteins are created in which mouse immunoglobulin constant regions are replaced with human immunoglobulin constant regions. See, e.g., Morrison et al. (1984) PROC. NAT. ACAD. SCI. USA 81:6851-6855, Neuberger et al., 1984, NATURE 312:604-608; U.S. Pat. Nos. 6,893,625; 5,500,362; and 4,816,567.

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of a subject antibody are grafted into human FRs or consensus human FRs. To create consensus human FRs, FRs from several human heavy chain or light chain amino acid sequences are aligned to identify a consensus amino acid sequence. CDR grafting is described in U.S. Pat. Nos. 7,022,500; 6,982,321; 6,180,370; 6,054,297; 5,693,762; 5,859,205; 5,693,761; 5,565,332; 5,585,089; 5,530,101; Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557; and Tan et al., 2002, J. IMMUNOL. 169:1119-1125.

Other methods to reduce immunogenicity include “reshaping,” “hyperchimerization,” and “veneering/resurfacing.” See, e.g., Vaswami et al. (1998) ANN. ALLERGY ASTHMA IMMUNOL. 81:105-115; Roguska et al. (1996) PROTEIN ENG. 9:895-904; and U.S. Pat. No. 6,072,035. In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641.

Another approach for converting a mouse antibody into a form suitable for medical use in humans is known as ACTIVMAB™ technology (Vaccinex, Inc., Rochester, N.Y.), which involves a vaccinia virus-based vector to express antibodies in mammalian cells. High levels of combinatorial diversity of IgG heavy and light chains are said to be produced. See, e.g., U.S. Pat. Nos. 6,706,477; 6,800,442; and 6,872,518.

Another approach for converting a mouse antibody into a form suitable for use in humans is technology practiced commercially by KaloBios Pharmaceuticals, Inc. (Palo Alto, Calif.). This technology involves the use of a proprietary human “acceptor” library to produce an “epitope focused” library for antibody selection.

Another approach for modifying a mouse antibody into a form suitable for medical use in humans is HUMAN ENGINEERING™ technology, which is practiced commercially by XOMA (US) LLC. See, e.g., PCT Publication No. WO 93/11794 and U.S. Pat. Nos. 5,766,886; 5,770,196; 5,821,123; and 5,869,619.

Any suitable approach, including any of the above approaches, can be used to reduce or eliminate human immunogenicity of an antibody.

In addition, it is possible to create fully human antibodies in non-human hosts, such as in rodents (for example, in mice), or via phage or yeast display libraries. For example, fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human mAbs can also be prepared and optimized from phage display libraries by techniques referenced in, e.g., Knappik et al., J. MOL. BIOL. 296:57-86, 2000; and Krebs et al., J. IMMUNOL. METH. 254:67-84 2001). In addition, fully human antibodies can be created de novo in yeast expression systems or murine antibodies can be humanized in yeast expression systems practiced commercially by Adimab LLC (Lebanon, N.H.). See, e.g., U.S Pat. Nos. 8,691,730 and 7,700,302, and U.S. Published Patent Application Nos. US2014/0221250, US2013/0197201, and US2012/0322672.

In each of the foregoing embodiments, it is contemplated herein that immunoglobulin heavy chain variable region sequences and/or light chain variable region sequences that together bind and may contain amino acid alterations (e.g., at least 1, 2, 3, 4, 5, or 10 amino acid substitutions, deletions, or additions) in the framework regions of the heavy and/or light chain variable regions.

### Exemplary Neutralizing Antibodies

An exemplary FSTL3 neutralizing antibody can be prepared as follows. It is understood that most of the binding energy of the FS-ligand interaction derives from the N-terminal domain (ND) of FS (Keutmann et al., (2004) MOLECULAR ENDOCRINOL. 18:228-240), and this is presumed to be true as well for FSTL3 (Cash et al., (2012) J. BIOL. CHEM. 287:1043-1053). From the structures of FS and FSTL3 in complex with activin and myostatin (Thompson et al., (2005) DEV. CELL 9:535-543; Stamler et al., (2008) J. BIOL. CHEM. 283:32831-32838; Cash et al., (2009) EMBO J. 28:2662-2676; Cash et al., (2012) J. BIOL. CHEM. 287:1043-1053), the key feature of the ND-ligand interaction is a long FS or FSTL3 α-helix that lies inside the “fingers” of the ligand. It is contemplated that an effective FSTL3 neutralizing antibody will bind this region. In order to prepare a monoclonal antibody of this specificity, one or more peptides encompassing the α-helical region (e.g. PGNKINLLGFLGLV (77-90) of human FSTL3 sequence (Uniprot accession 095633), which is 93% identical between both human and mouse, and human and rat) is chemically synthesized, conjugated to KLH, and then used to raise anti-peptide antibodies in an appropriate mouse host. Hybridomas are prepared by standard techniques, and screened, successively, for binding to the immunogen, binding to the native protein, and neutralizing the native protein in a reporter assay that can respond to GDF-11 signaling.

An exemplary GASP1 or GASP2 neutralizing antibody can be prepared as follows. It is understood that most of the binding energy of the GASP1 or GASP2-ligand interaction derives from the single follistatin-like domain (FSD) of GASP1 or GASP2 (Kondas et al., (2008) J. BIOL. CHEM. 283:23677-23684). There are no structures of GASP1 or 2 available, but an approximate structure for the GASP FSD can be derived by modeling based on the three FSDs in FS and the two in FSTL3. The first two FSDs in FS and FSTL3 contact the ligand, but, importantly, in different orientations. However, given that the GASP FSD can bind the latent complex, the only part of the ligand that is exposed in the latent complex (Shi et al., (2011) NATURE 474:343-349) corresponds to the region where the first FSD in FS and FSTL3 binds (Thompson et al., supra; Stamler et al., supra; Cash et al., (2009) supra; Cash et al., (2012) supra). Therefore a surface of the GASP FSD is chosen that corresponds to the surface of the first FS/FSTL3 FSD. To obtain monoclonal antibodies of this specificity, peptides encompassing the region are chemically synthesized. However, since FSDs have 5 Cys-Cys pairs per domain, the unpaired cysteine residues in the fragment are replaced by serines (e.g. FTsASDGLTYYNRsYMDAEAsSKGITLAVVT (144-174) of human GASP1 sequence (Uniprot accession Q8TEU8), which is 94% identical between both human and mouse, and human and rat, where the introduced serines are shown in lower case). The peptide is chemically synthesized, conjugated to KLH, and then used to raise anti-peptide antibodies in an appropriate mouse host. Hybridomas are prepared by standard techniques, and screened, successively, for binding to the immunogen, binding to the native protein, and neutralizing the native protein in a reporter assay that can respond to GDF-11 signaling.

The resulting FSTL3, GASP1 or GASP2 neutralizing antibodies can be humanized or converted to a corresponding human antibody to reduce immunogenicity using techniques used in the art, for example, those techniques discussed above.

### Small Molecule Antagonists

The methods of the invention can employ small molecules that inhibit endogenous negative regulators of GDF-11. Small molecules may have a molecular weight below 2,000 Daltons, more preferably between 300 and 1,000 Daltons, and most preferably between 400 and 700 Daltons. In particular embodiments, the small molecule is an organic molecule.

In addition, small molecule antagonists can be identified using methods known in the art. Screening assays to identify compounds that inhibit the activity of endogenous negative regulators of GDF-11 (e.g., FS, FSTL3, GASP1, GASP2, GDF-11 propeptide, or myostatin propeptide) can be carried out by standard methods. The screening methods may involve high-throughput techniques. In addition, these screening techniques may be carried out in cultured cells or in organisms such as worms, flies, or yeast.

Any number of methods is available for carrying out such screening assays. The effect of a candidate compound may be measured on polypeptide production. Here, candidate compounds are added at varying concentrations to the culture medium of cells expressing an endogenous negative regulator of GDF-11 and protein levels of the negative regulator are measured using, for example, standard immunological techniques, such as western blotting or immunoprecipitation with an antibody specific for the negative regulator. For example, immunoassays may be used to detect or monitor the expression of the negative regulator. Polyclonal or monoclonal antibodies which are capable of binding to such a polypeptide may be used in any standard immunoassay format (e.g., ELISA, western blot, or RIA assay) to measure the level of the negative regulator. In these assays, the level of protein expression in the presence of the candidate compound is compared to the level measured in a control culture medium lacking the candidate molecule. A compound which promotes a decrease in expression of the negative regulator is considered useful in the invention. Such a molecule may be used, for example, as a therapeutic for an age-related condition (e.g., diastolic heart failure).

In one embodiment, candidate compounds that affect binding of GDF-11 to an endogenous negative regulator of GDF-11 (e.g., any described herein) are identified. Disruption by a candidate compound of GDF-11 binding to the negative regulator may be assayed using methods standard in the art. Compounds that affect binding of GDF-11 to its endogenous negative regulator are considered compounds useful in the invention. Such compound may be used, for example, as a therapeutic in an age related condition (e.g., diastolic heart failure).

Alternatively, or in addition, candidate compounds may be screened for those which specifically bind to an endogenous negative regulator of GDF-11. The efficacy of such a candidate compound is dependent upon its ability to interact with the polypeptide. Such an interaction can be readily assayed using any number of standard binding techniques and functional assays (e.g., those described in Ausubel et al., Current Protocols in Molecular Biology, Wiley Interscience, New York, 1997). For example, a candidate compound may be tested in vitro for interaction and binding with the negative regulator, and its ability to modulate its activity may be assayed by any standard assay.

In one particular embodiment, a candidate compound that binds to an endogenous negative regulator of GDF-11 may be identified using a chromatography-based technique. For example, the recombinant negative regulator may be purified by standard techniques from cells engineered to express the negative regulator and may be immobilized on a column. A solution of candidate compounds is then passed through the column, and a compound specific for the negative regulator is identified on the basis of its ability to bind to the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). Compounds isolated by this approach may also be used, for example, as therapeutics to treat an age-related condition (e.g., diastolic heart failure). Compounds which are identified as binding to the negative regulator with an affinity constant less than or equal to 10 mM are considered particularly useful in the invention.

According to another approach, candidate compounds are added at varying concentrations to the culture medium of cells expressing a polynucleotide coding for an endogenous negative regulator of GDF-11. Gene expression is then measured, for example, by standard Northern blot analysis (Ausubel et al., supra), using any appropriate fragment prepared from the polynucleotide molecule as a hybridization probe. The level of gene expression in the presence of the candidate compound is compared to the level measured in a control culture medium lacking the candidate molecule. A compound which promotes an decrease in expression of the endogenous negative regulator is considered useful in the invention; such a molecule may be used, for example, as a therapeutic for an age-related condition (e.g., diastolic heart failure).

Optionally, compounds identified in any of the above-described assays may be confirmed as useful in delaying or ameliorating age-related conditions in either standard tissue culture methods or animal models and, if successful, may be used as therapeutics for treating age-related conditions (e.g., diastolic heart failure).

In general, compounds capable of treating an age-related condition (e.g., diastolic heart failure) are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and polynucleotide-based compounds. Synthetic compound libraries are commercially available. Alternatively, libraries of natural compounds in the form of bacterial, fungal, plant, and animal extracts are commercially available. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in treating an age-related condition (e.g., diastolic heart failure) should be employed whenever possible.

When a crude extract is found to have an activity that decreases expression or activity of an endogenous negative regulator of GDF-11, or a binding activity, further fractionation of the positive lead extract is necessary to isolate chemical constituents responsible for the observed effect. Thus, the goal of the extraction, fractionation, and purification process is the characterization and identification of a chemical entity within the crude extract having activity that may be useful in treating an age-related condition (e.g., diastolic heart failure). Methods of fractionation and purification of such heterogenous extracts are known in the art. If desired, compounds shown to be useful agents for the treatment of a age-related condition (e.g., diastolic heart failure) are chemically modified according to methods known in the art.

### Aptamer-Based Antagonists

The methods of the invention can also employ aptamer antagonists directed against an endogenous negative regulator of GDF-11 (e.g., FS, FSTL3, GASP1, GASP2, GDF-11 propeptide, or myostatin propeptide). Aptamers, also known as nucleic acid ligands, are non-naturally occurring nucleic acids that bind to and, generally, antagonize (i.e inhibit) a pre-selected target.

Particular examples of such aptamers include the FS- and FSTL3-binding aptamers created by SomaLogic (sequence IDs 4132-27_2 and 3438-10_2, respectively).

Aptamers can be made by any known method of producing oligomers or oligonucleotides. Many synthesis methods are known in the art. For example, 2′-O-allyl modified oligomers that contain residual purine ribonucleotides, and bearing a suitable 3′-terminus such as an inverted thymidine residue (Ortigao et al., ANTISENSE RES. DEV. 2:129-146 (1992)) or two phosphorothioate linkages at the 3′-terminus to prevent eventual degradation by 3′-exonucleases, can be synthesized by solid phase beta-cyanoethyl phosphoramidite chemistry (Sinha et al., NUCLEIC ACIDS RES. 12:4539-4557 (1984)) on any commercially available DNA/RNA synthesizer. One method is the 2′-O-tert-butyidimethylsilyl (TBDMS) protection strategy for the ribonucleotides (Usman el al., J. AM. CHEM. SOC. 109:7845-7854 (1987)), and all the required 3′-O-phosphoramidites are commercially available. In addition, aminomethylpolystyrene may be used as the support material due to its advantageous properties (McCollum and Andrus (1991) TETRAHEDRON LETT. 32:4069-4072). Fluorescein can be added to the 5′-end of a substrate RNA during the synthesis by using commercially available fluorescein phosphoramidites. In general, an aptatner oligomer can be synthesized using a standard RNA cycle. Upon completion of the assembly, all base labile protecting groups are removed by an eight hour treatment at 55° C. with concentrated aqueous ammonia/ethanol (3:1 v/v) in a sealed vial. The ethanol suppresses premature removal of the 2′-O-TBDMS groups that would otherwise lead to appreciable strand cleavage at the resulting ribonucleotide positions under the basic conditions of the deprotection (Usman et al., (1987) J. AM. CHEM. SOC. 109:7845-7854). After lyophilization, the TBDMS protected oligomer is treated with a mixture of triethylamine trihydrofluoride/triethylamine/N-methylpyrrolidinone for 2 hours at 60° C. to afford fast and efficient removal of the silyl protecting groups under neutral conditions (see Wincott et al., (1995) NUCLEIC ACIDS RES. 23:2677-2684). The fully deprotected oligomer can then be precipitated with butanol according to the procedure of Cathala and Brunel ((1990) NUCLEIC ACIDS RES. 18:201). Purification can be performed either by denaturing polyacrylamide gel electrophoresis or by a combination of ion-exchange HPLC (Sproat el al., (1995) NUCLEOSIDES NUCLEOTIDES 14:255-273) and reversed phase HPLC. For use in cells, synthesized oligomers are converted to their sodium salts by precipitation with sodium perchlorate in acetone. Traces of residual salts may then be removed using small disposable gel filtration columns that are commercially available. As a final step the authenticity of the isolated oligomers may be checked by matrix assisted laser desorption mass spectrometry (Pieles et al., (1993) NUCLEIC ACIDS RES. 21:3191-3196) and by nucleoside base composition analysis.

Aptamers can also be produced through enzymatic methods, when the nucleotide subunits are available for enzymatic manipulation. For example, the RNA molecules can be made through in vitro RNA polymerase T7 reactions. They can also be made by strains of bacteria or cell lines expressing T7, and then subsequently isolated from these cells. As discussed below, the disclosed aptamers can also be expressed in cells directly using vectors and promoters.

The aptamers may further contain chemically modified nucleotides. One issue to be addressed in the diagnostic or therapeutic use of nucleic acids is the potential rapid degradation of oligonucleotides in their phosphodiester form in body fluids by intracellular and extracellular enzymes such as endonucleases and exonucleases before the desired effect is manifest. Certain chemical modifications of the nucleic acid ligand can be made to increase the in vivo stability of the nucleic acid ligand or to enhance or to mediate the delivery of the nucleic acid ligand (see, e.g., U.S. Pat. No. 5,660,985, entitled “High Affinity Nucleic Acid Ligands Containing Modified Nucleotides”).

Modifications of the nucleic acid ligands contemplated in this invention include, but are not limited to, those which provide other chemical groups that incorporate additional charge, polarizability, hydrophobicity, hydrogen bonding, electrostatic interaction, and fluxionality to the nucleic acid ligand bases or to the nucleic acid ligand as a whole. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil; backbone modifications, phosphorothioate or alkyl phosphate modifications, methylations, unusual base-pairing combinations such as the isobases isocytidine and isoguanidine and the like. Modifications can also include 3′ and 5′ modifications such as capping or modification with sugar moieties. In some embodiments of the instant invention, the nucleic acid ligands are RNA molecules that are 2′-fluoro (2′-F) modified on the sugar moiety of pyrimidine residues.

The stability of the aptamer can be greatly increased by the introduction of such modifications and as well as by modifications and substitutions along the phosphate backbone of the RNA. In addition, a variety of modifications can be made on the nucleobases themselves which both inhibit degradation and which can increase desired nucleotide interactions or decrease undesired nucleotide interactions. Accordingly, once the sequence of an aptamer is known, modifications or substitutions can be made by the synthetic procedures described below or by procedures known to those of skill in the art.

Other modifications include the incorporation of modified bases (or modified nucleoside or modified nucleotides) that are variations of standard bases, sugars and/or phosphate backbone chemical structures occurring in ribonucleic (i.e., A, C, G and U) and deoxyribonucleic (i.e., A, C, G and T) acids. Included within this scope are, for example: Gm (2′-methoxyguanylic acid), Am (2′-methoxyadenylic acid), Cf (2′-fluorocytidylic acid), Uf (2′-fluorouridylic acid), Ar (riboadenylic acid). The aptamers may also include cytosine or any cytosine-related base including 5-methylcytosine, 4-acetylcytosine, 3-methylcytosine, 5-hydroxymethyl cytosine, 2-thiocytosine, 5-halocytosine (e.g., 5-fluorocytosine, 5-bromocytosine, 5-chlorocytosine, and 5-iodocytosine), 5-propynyl cytosine, 6-azocytosine, 5-trifluoromethylcytosine, N4, N4-ethanocytosine, phenoxazine cytidine, phenothiazine cytidine, carbazole cytidine or pyridoindole cytidine. The aptamer may further include guanine or any guanine-related base including 6-methylguanine, 1-methylguanine, 2,2-dimethylguanine, 2-methylguanine, 7-methylguanine, 2-propylguanine, 6-propylguanine, 8-haloguanine (e.g., 8-fluoroguanine, 8-bromoguanine, 8-chloroguanine, and 8-iodoguanine), 8-aminoguanine, 8-sulfhydrylguanine, 8-thioalkylguanine, 8-hydroxylguanin 7-methylguanine, 8-azaguanine, 7-deazaguanine or 3-deazaguanine.

Also included are the modified nucleobases described in U.S. Pat. Nos. 3,687,808; 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; 5,645,985; 5,830,653; 5,763,588; 6,005,096; and 5,681,941. Examples of modified nucleoside and nucleotide sugar backbone variants known in the art include, without limitation, those having, e.g., 2′ ribosyl substituents such as F, SH, SCH₃, OCN, Cl, Br, CN, CF₃, OCF₃, SOCH₃, SO₂, CH₃, ONO₂, NO₂, N₃, NH₂, OCH₂CH₂OCH₃, O(CH₂)₂ON(CH₃)₂, OCH₂OCH₂N(CH₃)₂, O(C₁₋₁₀ alkyl), O(C₂₋₁₀ alkenyl), O(C₂₋₁₀ alkynyl), S(C₁₋₁₀ alkyl), S(C₂₋₁₀ alkenyl), S(C₂₋₁₀ alkynyl), NH(C₁₋₁₀ alkyl), NH(C₂₋₁₀ alkenyl), NH(C₂₋₁₀ alkynyl), and O-alkyl-O-alkyl. Desirable 2′ ribosyl substituents include 2′-methoxy (2′-OCH₃), 2′-aminopropoxy (2′ OCH₂CH₂CH₂NH₂), 2′-allyl (2′-CH₂—CH═CH₂), 2′-O-allyl(2′-O—CH₂—CH═CH₂), 2′-amino (2′-NH₂), and 2′-fluoro (2′-F). The 2′-substituent may be in the arabino (up) position or ribo (down) position.

The aptamers of the invention may be made up of nucleotides and/or nucleotide analogs such as described above, or a combination of both, or are oligonucleotide analogs. The aptamers of the invention may contain nucleotide analogs at positions which do not affect the function of the oligomer to bind the endogenous negative regulator of GDF-11.

There are several techniques that can be adapted for refinement or strengthening of the nucleic acid ligands binding to a particular target molecule or the selection of additional aptamers. One technique, generally referred to as “in vitro genetics” (see Szostak (1992) TRENDS BIOCHEM. SCI. 17:89-93), involves isolation of aptamer antagonists by selection from a pool of random sequences. The pool of nucleic acid molecules from which the disclosed aptamers may be isolated may include invariant sequences flanking a variable sequence of approximately twenty to forty nucleotides. This method has been termed Selective Evolution of Ligands by EXponential Enrichment (SELEX). Compositions and methods for generating aptamer antagonists of the invention by SELEX and related methods are known in the art and taught in, for example, U.S. Pat. Nos. 5,475,096 and 5,270,163. The SELEX process in general are further described in, e.g., U.S. Pat. Nos. 5,668,264; 5,696,249; 5,670,637; 5,674,685; 5,723,594; 5,756,91; 5,811,533; 5,817,785; 5,958,691; 6,011,020 6,051,698; 6,147,204; 6,168,778; 6,207,816; 6,229,002; 6,426,335; and 6,582,918.

Briefly, the SELEX method involves selection from a mixture of candidate oligonucleotides and step-wise iterations of binding to a selected target, partitioning and amplification, using the same general selection scheme, to achieve virtually any desired criterion of binding affinity and selectivity. Starting from a mixture of nucleic acids, typically comprising a segment of randomized sequence, the SELEX method includes steps of contacting the mixture with the target under conditions favorable for binding, partitioning unbound nucleic acids from those nucleic acids which have bound specifically to target molecules, dissociating the nucleic acid-target complexes, amplifying the nucleic acids dissociated from the nucleic acid-target complexes to yield a ligand-enriched mixture of nucleic acids, then reiterating the steps of binding, partitioning, dissociating and amplifying through as many cycles as desired to yield highly specific high affinity nucleic acid ligands to the target molecule.

Aptamers with these various modifications can then be tested for function using any suitable assay. The modifications can be pre- or post-SELEX process modifications. Pre-SELEX process modifications yield nucleic acid ligands with both specificity for their SELEX target and improved in vivo stability. Post-SELEX process modifications made to 2′-OH nucleic acid ligands can result in improved in vivo stability without adversely affecting the binding capacity of the nucleic acid ligand. Other modifications useful for producing aptamers of the invention are known to one of ordinary skill in the art. Such modifications may be made post-SELEX process (modification of previously identified unmodified ligands) or by incorporation into the SELEX process.

### Antisense Inhibitors

In certain embodiments, the compounds that inhibit the activity of endogenous negative regulators of GDF-11 (e.g., FS, FSTL3, GASP1, GASP2, GDF-11 propeptide, or myostatin propeptide) is an antisense therapeutic, such RNA interference (RNAi) molecule or a small interfering RNA (siRNA). Such compounds may be synthesized by any of the known chemical oligonucleotide and peptidyl nucleic acid synthesis methodologies known in the art (see, for example, PCT/EP92/20702 and PCT/US94/013523) and used in antisense therapy. Anti-sense oligonucleotide and peptidyl nucleic acid sequences, usually 10 to 100 and more preferably 15 to 50 units in length, are capable of hybridizing to a gene and/or mRNA transcript and, therefore, may be used to inhibit transcription and/or translation of a target protein. Gene expression of endogenous negative regulators of GDF-11 (e.g., FS, FSTL3, GASP1, GASP2, GDF-11 propeptide, or myostatin propeptide) therefore can be inhibited by using nucleotide sequences complementary to a regulatory region of any of these genes (e.g., the promoter and/or a enhancer) to form triple helical structures that prevent transcription of any of these gene in target cells. See generally, Helene (1991) Anticancer Drug Des. 6(6): 569-84, Helene et al. (1992) i Ann. N.Y. Acad. Sci. 660: 27-36; and Maher (1992) Bioessays 14(12): 807-15.

The antisense sequences may be modified at a base moiety, sugar moiety or phosphate backbone to improve, e.g., the stability, hybridization, or solubility of the molecule. For example, in the case of nucleotide sequences, phosphodiester linkages may be replaced by thioester linkages making the resulting molecules more resistant to nuclease degradation. Alternatively, the deoxyribose phosphate backbone of the nucleic acid molecules can be modified to generate peptide nucleic acids (see Hyrup et al. (1996) Bioorg. Med. Chem. 4(1): 5-23). Peptidyl nucleic acids have been shown to hybridize specifically to DNA and RNA under conditions of low ionic strength. Furthermore, it is appreciated that the peptidyl nucleic acid sequences, unlike regular nucleic acid sequences, are not susceptible to nuclease degradation and, therefore, are likely to have greater longevity in vivo. Furthermore, it has been found that peptidyl nucleic acid sequences bind complementary single stranded DNA and RNA strands more strongly than corresponding DNA sequences (PCT/EP92/20702). Similarly, oligoribonucleotide sequences generally are more susceptible to enzymatic attack by ribonucleases than are deoxyribonucleotide sequences, such that oligodeoxyribonucleotides are likely to have greater longevity than oligoribonucleotides for in vivo use.

Additionally, RNAi can serve as a treatment agent. To the extent RNAi is used, double stranded RNA (dsRNA) having one strand identical (or substantially identical) to the target mRNA sequence (e.g. an endogenous negative regulator of GDF-11 such as FS, FSTL3, GASP1, GASP2, GDF-11 propeptide, or myostatin propeptide) is introduced to a cell. The dsRNA is cleaved into small interfering RNAs (siRNAs) in the cell, and the siRNAs interact with the RNA induced silencing complex to degrade the target mRNA, ultimately destroying production of a desired gene product. Alternatively, the siRNA can be introduced directly. RNAi can be used as an antagonist against endogenous negative regulator of GDF-11 (e.g., FS, FSTL3, GASP1, GASP2, GDF-11 propeptide, or myostatin propeptide).

Specific examples of FS antisense molecules include those from Novus Biologicals, (cat. #H00010468-R01 and H00010468-R02 and those from Santa Cruz Biotechnology (cat. #sc-39762). Specific examples of FSTL3 antisense molecules include those from Origene (cat. No. SR306954) and those from Qiagen (Cat. #SI00422009; SI03019247; SI03019548; SI04230100; SI04242623; SI04296005). Specific examples of GASP1 antisense molecules include those from Santa Cruz Biotechnology (cat. #.sc-90993) and those from Novus Biologicals (Cat. #H00009737-R02). Specific examples of GASP2 antisense molecules include those from Santa Cruz Biotechnology (cat. #sc-91285), those from Abnova (Cat. #H00114928-R03 and H00114928-R04), and Origene (cat. #SR314476).

### Disease Indications

The antagonists disclosed herein can be used to treat a variety of disorders associated with aging, for example, cardiovascular disorders, cognitive disorders, neurodegenerative disorders, metabolic disorders, or muscular disorders.

Age related cardiovascular disorders, include, for example, diastolic heart failure, angina, atherosclerosis, coronary artery disease, congestive heart failure, hypertension and atrial fibrillation.

Age related cognitive disorders, include, for example, memory deficits associated with ageing, schizophrenia, special learning disorders, seizures, post-stroke convulsions, brain ischemia, hypoglycemia, cardiac arrest, epilepsy, as well as Huntington's, Parkinson's and Alzheimer's disease.

Age related neurodegenerative disorders include, for example, Alzheimer's disease, Parkinson's disease, Amyotrophic lateral sclerosis (ALS), motor neuron disease, ischemic stroke, Huntington's disease, multiple sclerosis, Pick's disease, fronto-temporal dementia, cortico-basal degeneration, progressive supranuclear palsy, Creutzfeldt-Jakob disease, and Gerstmann-Straussler-S cheinker syndrome.

Age related metabolic disorders include, for example, type II diabetes, metabolic syndrome, hyperglycemia, hypercholesterolemia, hyperlipidemia, and obesity.

Age related muscular disorders include, for example, sarcopenia, and disuse atrophy.

A preferred age related cardiovascular disorder is diastolic heart failure, a clinical syndrome that occurs in a variety of pathophysiologic settings, including long-standing hypertension, valvular disease such as aortic stenosis, genetic hypertrophic cardiomyopathy, and as a result of aging. These disparate etiologies converge with some common pathophysiologic threads, most obviously with cellular hypertrophy or increased diameter of cardiomyocytes, which translates into increased thickness of the heart wall without significantly reducing squeezing capacity (systolic function). Myocardial hypertrophy is an important contributor to the impairment in relaxation or increased stiffness that causes diastolic heart failure (Wagers et al. (2002) SCIENCE 297:2256-2259).

During aging, cardiac tissues often experience a decrease in diastolic function related to a thickening and/or stiffening of the tissue or cardiac hypertrophy. As used herein, the term “cardiac hypertrophy” as used herein refers to an enlargement of the heart due in part to an increase in the size of the myocytes. In some embodiments, the myocytes respond to stress through hypertrophic growth. Cardiac hypertrophy is often associated with increased risk of morbidity and mortality. In some embodiments, the cardiac hypertrophy is left ventricle cardiac hypertrophy. The term “left ventricle cardiac hypertrophy” as used herein refers to a disorder in which the myocardial tissue of the left ventricle of the heart thickens. Without wishing to be bound by theory, causes of left ventricle cardiac hypertrophy include, for example, hypertension (e.g., high blood pressure), stenosis of the aortic valve (e.g., the inability of the heart valve to fully open), and hypertrophic cardiomyopathy (e.g., a disorder in which the myocardial tissue thickens for no obvious cause). In other embodiments, the cardiac hypertrophy is right ventricle cardiac hypertrophy. The term “right ventricle cardiac hypertrophy” as used herein refers to a disorder in which the myocardial tissue of the right ventricle thickens. Without wishing to be bound by theory, causes of right ventricle hypertrophy include, for example, diseases that damage the lungs (e.g., such as emphysema and cystic fibrosis), conditions that decrease oxygen levels in the body (e.g., chronic bronchitis and sleep apnea), stenosis of the pulmonic heart valve, chronic pulmonary embolism, primary pulmonary hypertension, asymmetric septal hypertrophy, and idiopathic hypertrophic subaortic stenosis.

Symptoms of cardiac hypertrophy and methods of measuring them are well known in the art and include but are not limited to, an increase in left ventricular mass, a change in body weight ratio, a change in cardiomyocyte size or mass, a change in cardiomyocyte organization, changes in cardiac gene expression, changes in cardiac function (e.g., diastolic heart function), fibroid deposition, changes in dP/dT (rate of change of the ventricular pressure with respect to time), calcium ion flux, stroke length, and ventricular output. Diagnostic procedures useful in detecting cardiovascular conditions and/or efficacy of treatment of cardiovascular conditions include echocardiography (e.g., 2 and 3 dimensional), MRI (e.g., spin-echo MRI or cine magnetic resonance angiography), chest radiography, thallium-201 myocardial imaging, PET, ECG-gated CT, cardiac catheterization, angiography, electrophysiological studies, and magnetic resonance spectroscopy. For example, echocardiography can detect the size of the heart, the pattern of hypertrophy, the contractile function of the heart, and the severity of the outflow gradient while MRI can evaluate ventricular anatomy, wail thickness, ventricular function, ventricular end-diastolic and end-systolic volumes, valvular dysfunction, and outflow tract obstruction.

Neurodegenerative disease is marked by neuronal loss, often of specific cell types, e.g., dopaminergic neurons in Parkinson's disease, cholinergic neurons in Alzheimer's diseases, and orexigenic neurons in narcolepsy with cataplexy. In other cases, such as stroke, the effects are geographically restricted. Neurons of the CNS generally show little regenerative capacity, except for the subventricular zone, which harbors the neural stem cell niche. Effective promotion of neuronal stem cell proliferation leads to repopulation of the missing neurons of the CNS and for degenerative conditions would prevent further decline or even reverse functional loss, as measured by movement in the case of Parkinson's disease, memory and cognition in the case of Alzheimer's disease, and improved sleep regulation in the case of narcolepsy with cataplexy. For stroke, which is non-degenerative, effective promotion of neuronal stem cell proliferation would lead to improved movement or speech function, depending on the geographic area of the brain affected.

Disuse atrophy of skeletal muscle can occur at any age, but occurs more rapidly in the elderly. An elderly person on bed rest can lose 1-2 kg of muscle mass per week, which can create significant co-morbidities and impair full ambulatory recovery. In this population, effective skeletal muscle regeneration, including neuromuscular junction function, would be expected to allow more complete and rapid recovery following a period of bed rest, such as after hip replacement surgery.

### Dosage and Administration

Generally, a therapeutically effective amount of an antagonist of a negative regulator of GDF-11 (e.g., an antibody) is in the range of 0.1 mg/kg to 100 mg/kg, e.g., 1 mg/kg to 100 mg/kg, e.g., 1 mg/kg to 10 mg/kg, e.g., 2.0 mg/kg to 10 mg/kg. However, the amount of an antagonist of a negative regulator administered will depend on variables such as the type and extent of disease or indication to be treated, the overall health of the patient, the in vivo potency of the antagonist, the pharmaceutical formulation, the serum half-life of the antagonist, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from, for example, 0.5 mg/kg to 20 mg/kg in the case of an antibody-based antagonist. Dosing frequency can vary, depending on factors such as route of administration, dosage amount, serum half-life of the antagonist, and the disease being treated. Exemplary dosing frequencies are once per day, once per week and once every two weeks. In some embodiments, dosing is once every two weeks.

For therapeutic use, an antagonist preferably is formulated with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Pharmaceutical compositions containing antagonists of endogenous negative regulators of GDF-11 (e.g., antibodies), such as those disclosed herein, can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyetheylene glycol), and suitable mixtures thereof.

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

### EXAMPLES

The following Examples are merely illustrative and are not intended to limit the scope or content of the invention in any way.

### Example 1

### FSTL3-Neutralizing Antibodies

Most of the binding energy of the FS-ligand interaction derives from the N-terminal domain (ND) of FS (Keutmann et al., (2004) MOLECULAR ENDOCRINOL. 18:228-240), and this is presumed to be true as well for FSTL3 (Cash et al., (2012) J. BIOL. CHEM. 287:1043-1053). From the structures of FS and FSTL3 in complex with activin and myostatin (Thompson et al., (2005) DEV. CELL 9:535-543; Stamler et al., (2008) J. BIOL. CHEM. 283:32831-32838; Cash et al., (2009) EMBO J. 28:2662-2676; Cash et al., (2012) J. BIOL. CHEM. 287:1043-1053), the key feature of the ND-ligand interaction is a long FS or FSTL3 α-helix that lies inside the “fingers” of the ligand. Therefore an effective FSTL3 neutralizing monoclonal antibody targets this region. To obtain monoclonal antibodies of this specificity, peptides encompassing the α-helical region (e.g. PGNKINLLGFLGLV (77-90) of human FSTL3 sequence (Uniprot accession 095633), which is 93% identical between both human and mouse, and human and rat) are chemically synthesized, conjugated to KLH, and then used to raise anti-peptide antibodies in an appropriate mouse host. Hybridomas are prepared by standard techniques, and screened, successively, for binding to the immunogen, binding to the native protein, and neutralizing the native protein in a reporter assay that can respond to GDF-11 signaling.

The resulting antibody can be humanized or converted to a human antibody to reduce immunogenicity using techniques used in the art.

### Example 2

### GASP1 or GASP2-Neutralizing Antibodies

Most of the binding energy of the GASP1 or GASP2-ligand interaction derives from the single follistatin-like domain (FSD) of GASP1 or GASP2 (Kondas et al., (2008) J. BIOL. CHEM. 283:23677-23684). There are no structures of GASP1 or 2 available, but an approximate structure for the GASP FSD can be derived by modeling based on the three FSDs in FS and the two in FSTL3. The first two FSDs in FS and FSTL3 contact the ligand, but, importantly, in different orientations. However, given that the GASP FSD can bind the latent complex, the only part of the ligand that is exposed in the latent complex (Shi et al., (2011) NATURE 474:343-349) corresponds to the region where the first FSD in FS and FSTL3 binds (Thompson et al., supra; Stamler et al., supra; Cash et al., (2009) supra; Cash et al., (2012) supra). Therefore a surface of the GASP FSD is chosen that corresponds to the surface of the first FS/FSTL3 FSD. To obtain monoclonal antibodies of this specificity, peptides encompassing the region are chemically synthesized. However, since FSDs have 5 Cys-Cys pairs per domain, the unpaired cysteine residues in the fragment are replaced by serines (e.g. FTsASDGLTYYNRsYMDAEAsSKGITLAVVT (144-174) of human GASP1 sequence (Uniprot accession Q8TEU8), which is 94% identical between both human and mouse, and human and rat). The peptide is chemically synthesized, conjugated to KLH, and then used to raise anti-peptide antibodies in an appropriate mouse host. Hybridomas are prepared by standard techniques, and screened, successively, for binding to the immunogen, binding to the native protein, and neutralizing the native protein in a reporter assay that can respond to GDF-11 signaling.

The resulting antibody can be humanized or converted to a human antibody to reduce immunogenicity using techniques used in the art.

### Example 3

### Use of a Neutralizing Monoclonal Antibody Against an Endogenous Negative Regulator of GDF-11

The efficacy of monoclonal antibodies obtained in Examples 1 or 2 is demonstrated in either naturally aged animals (≧20 months in the case of rodents), or in models of age-related disease, such as the uninephrectomy/DOCA salt model in the rat, which causes diastolic heart failure. In naturally aged animals, a number of age-dependent processes are evaluated, including cardiac hypertrophy, neural stem cell proliferation, muscle satellite cell proliferation, neuromuscular junction morphology, and skeletal muscle function.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and the range of equivalency of the claims are intended to be embraced therein.

What is claimed is:

1. A method of treating an age-related condition in a subject in need thereof, the method comprising administering an effective amount of an antagonist of an endogenous negative regulator of GDF-11 activity to the subject, thereby ameliorating at least one symptom of the age-related condition in the subject.

2. The method of claim 1, wherein the age-related condition is a cardiovascular disorder, a cognitive disorder, a neurodegenerative disorder, a metabolic disorder, or a muscular disorder.

3. A method of ameliorating at least one symptom of diastolic heart failure in a subject in need thereof, the method comprising administering an effective amount of an antagonist of an endogenous negative regulator of GDF-11 activity to the subject, thereby ameliorating at least one symptom of the diastolic heart failure in the subject.

4. The method of claim 3, wherein the subject has preserved ejection fraction but elevated left ventricular diastolic pressure (LVDP) compared to subjects without diastolic heart failure.

5. The method of claim 3, wherein the subject has preserved ejection fraction but elevated myocardial fibrosis compared to subjects without diastolic heart failure.

6. The method of claim 3, wherein administration of the antagonist reduces left ventricle wall thickness, myocardial fibrosis, or both left ventricle wall thickness and myocardial fibrosis in the subject.

7. A method of treating diastolic heart failure in a subject in need thereof, the method comprising administering an effective amount of an antagonist of an endogenous negative regulator of GDF-11 activity to the subject, thereby treating diastolic heart failure in the subject.

8. The method claim 7, wherein the administration of the antagonist reduces left ventricle wall thickness in the subject relative to left ventricle wall thickness prior to initial administration of the antagonist.

9. The method of claim 7, wherein the administration of the antagonist reduces myocardial fibrosis in the subject relative to myocardial fibrosis prior to initial administration of the antagonist.

10. The method of claim 6, wherein the myocardial fibrosis is caused by an accumulation of extracellular matrix fibrillar collagen, and the administration of the antagonist reduces the amount of extracellular matrix fibrillar collagen in the subject relative to before initial administration of the antagonist.

11. The method of claim 1, wherein the endogenous negative regulator of GDF-11 activity is FS, FSTL3, GASP1, GASP2, GDF-11 propeptide, or myostatin propeptide.

12. The method of claim 1, wherein the antagonist is a protein.

13. The method of claim 12, wherein the protein is an antibody.

14. The method of claim 1, wherein the antagonist is an anti-FS antibody, an anti-FSTL3 antibody, an anti-GASP1 antibody, an anti-GASP2 antibody, an anti-GDF-11 propeptide antibody or an anti-myostatin propeptide antibody.

15. The method of claim 1, wherein the antagonist is an anti-GASP1 antibody.

16. The method of claim 1, wherein the antagonist is an anti-GASP2 antibody.

17. The method of claim 1, wherein the antagonist is an anti-FS antibody.

18. The method of claim 1, wherein administration of the antagonist does not cause a substantial (i) reduction of skeletal muscle mass of the subject, (ii) reduction of erythropoiesis in the subject, (iii) increase in follicle-stimulating hormone (FSH) activity in the subject, or (iv) a combination thereof.

19. The method claim 18, wherein administration of the antagonist does not cause anosmia in the subject.

20-23. (canceled)
